(19) United States
(12) Patent Application Publication
(10) Pub. No.: US 2022/0046883 A1
(43) Pub. Date: Feb. 17, 2022

(54) Methods and Compositions for Producing Cannabinoids

(71) Applicant: PLURISTEM LTD., Haifa (IL)

(72) Inventors: Yaacob YANAY, Shimshit (IL); Lior RAVIV, Kfar Monash (IL)

(73) Assignee: PLURISTEM LTD., Haifa (IL)

(21) Appl. No.: 17/414,328

(22) PCT Filed: Apr. 20, 2020
(86) PCT No.: PCT/IL2020/050477

§ 371 Date: Jun. 16, 2021

Related U.S. Application Data
(60) Provisional application No. 62/839,714, filed on Apr. 28, 2019.

(51) Int. Cl. A01H 6/28; C12M 1/12; C12M 1/08; A01H 4/00

(52) U.S. Cl. CPC A01H 6/28; C12M 25/06; A01H 4/00; C12M 27/24; C12M 25/16

(57) ABSTRACT

Disclosed herein are methods of producing one or more cannabinoid compounds, expanding cells that produce the compounds, cannabinoid compounds produced by the methods, pharmaceutical compositions comprising the cannabinoid compounds, and methods of producing and utilizing the compounds and compositions.

### FIELD

Disclosed herein are methods of producing cannabinoids and expanding cannabinoid-producing cells.

### BACKGROUND

Cannabinoids are a class of specialized compounds synthesized by Cannabis. They are formed by condensation of terpene and phenol precursors. They include these more abundant forms: Delta-9-tetrahydrocannabinol (THC), cannabidiol (CBD), cannabichromene (CBC), and cannabigerol (CBG). Another cannabinoid, cannabinol (CBN), is formed from THC as a degradation product and can be detected in some plant strains. Typically, THC, CBD, CBC, and CBG occur together in different ratios in various plant strains.

Cannabinoids are widely consumed, in a variety of forms around the world. Cannabinoid-rich preparations of Cannabis, either in herb (i.e., marijuana) or resin form (i.e., hash oil), are used by an estimated 2.6-5.0% of the world population (UNODC, 2012). Cannabinoid containing pharmaceutical products, either containing natural cannabis extracts (Sativex®) or the synthetic cannabinoids dronabinol or nabilone, are available for medical use in several countries. Δ-9-tetrahydrocannabinol (also known as THC) is one of the main biologically active components in the Cannabis plant, and it has been approved by the U.S. Food and Drug Administration (FDA) for the control of nausea and vomiting associated with chemotherapy and for appetite stimulation of AIDS patients suffering from wasting syndrome. The drug, however, shows other biological activities that lend themselves to possible therapeutic applications, such as treatment of glaucoma, migraine headaches, spasticity, anxiety, and as an analgesic.

Traditional methods of cannabinoid production typically focus on extraction and purification of cannabinoids from raw harvested Cannabis. However, traditional cannabinoid extraction and purification methods have a number of technical and practical problems that limits their usefulness—for example, difficulty in maintaining strain integrity, variable yields, due to pests and other natural causes, contamination with pesticides, and limited arable land area.

Production of cannabinoids, endocannabinoids, and related compounds via culture-based systems has the potential to overcome many, if not all, of the aforementioned issues. However, development of such culture systems is a field in its infancy. WO 2018/176055 and WO 2019/014395, both in the name of Trait Biosciences, generally discuss 1) cells transfected with constructs encoding cannabinoid synthetases, and related enzymes, and their use in synthesis of cannabinoids; and 2) methods of derivatizing cannabinoid compounds to increase their water solubility; however, these documents do not provide sufficient practical information to produce cannabinoids, or related compounds, via cell culture-based systems.

Furthermore, existing methods are difficult or impossible to adapt to GMP requirements and/or a quite limiting in the number of cycles per year that can be performed, thus limiting production ability.

### SUMMARY

Provided herein are methods of producing one or more cannabinoid compounds, comprising incubating plant cells in a nutrient medium, inside a bioreactor.

In certain embodiments, the cells are adherent cells. In more specific embodiments, the cells are cultured on a 2-dimensional (2D) substrate, a 3-dimensional (3D) substrate, or a 2D substrate, followed by a 3D substrate. Non-limiting examples of 2D and 3D substrates are provided in the Detailed Description and Examples.

The terms “two-dimensional culture”, “2D culture”, and “two-dimensional [or 2D] substrate” refer to a culture in which the cells are exposed to conditions that are compatible with cell growth and allow the cells to grow in a monolayer, which is referred to as a “two-dimensional culture apparatus”. Such apparatuses will typically have flat growth surfaces, in some embodiments comprising an adherent material, which may be planar or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture”, “3D culture”, and “three-dimensional [or 3D] substrate” refer to a culture in which the cells are exposed to conditions that are compatible with cell growth and allow the cells to grow in a 3D orientation (for example, outside of the plane of a monolayer) relative to one another. The term “three-dimensional [or 3D] culture apparatus” refers to an apparatus for culturing cells, which are, in some embodiments, adherent cells, under conditions that are compatible with cell growth and allow the cells to grow in a 3D orientation relative to one another. Such apparatuses will typically have a 3D growth surface, in some embodiments comprising an adherent material.

In general, reference to cell “growth” and “expansion” may be used interchangeably herein.

Except where otherwise indicated, all ranges mentioned herein are inclusive.

Except where otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to culture cannabinoid-producing cells.

FIG. 2A is a perspective view of a carrier (or “3D body”), according to an exemplary embodiment. B is a perspective view of a carrier, according to another exemplary embodiment. C is a cross-sectional view of a carrier, according to an exemplary embodiment.

FIG. 3 is a schematic depiction of synthesis of cannabinoids and enzymes involved therein, showing by-products (A) and intended products (B).

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Provided herein are methods of producing one or more cannabinoid compounds, comprising incubating plant cells in a nutrient medium, inside a bioreactor.

In some embodiments, there is a provided a method of producing one or more cannabinoid compounds, comprising: incubating plant cells in a production medium, inside a bioreactor, under conditions where the plant cells produce the cannabinoid compound(s); thereby producing cannabinoid compound(s). In certain embodiments, the plant cells secrete the cannabinoid compound(s) into the production medium; and/or the cannabinoid compound(s) are removed from the production medium on an ongoing basis. Optionally, an additional step of (b) obtaining the production medium from the bioreactor is performed; e.g., for obtaining residual cannabinoids not recovered during the previous steps. In certain embodiments, the plant cells both synthesize and secrete the cannabinoids, while inside the bioreactor.

In other embodiments, there is a provided a method of producing a composition comprising one or more cannabinoid compounds, comprising: incubating plant cells in a production medium, inside a bioreactor, under conditions where the plant cells produce the cannabinoid compound(s); thereby producing cannabinoid compound(s). In certain embodiments, the plant cells secrete the cannabinoid compound(s) into the production medium; and/or the cannabinoid compound(s) are removed from the production medium on an ongoing basis. In certain embodiments, the cannabinoid compound(s) are removed from the production medium on an ongoing basis, in some embodiments together with other compounds that co-purify therewith. In some embodiments, there is an additional step of (b) obtaining the production medium from the bioreactor at the conclusion of the incubation; e.g. for obtaining residual cannabinoids not recovered during the previous steps. In certain embodiments, the plant cells both synthesize and secrete the cannabinoids, while inside the bioreactor.

In still other embodiments, there is provided a method of expanding a population of cannabinoid-producing cells, comprising: incubating the cannabinoid-producing cells in a production medium, inside a bioreactor, under conditions where the plant cells produce the cannabinoid compound(s); thereby expanding a population of cannabinoid-producing cells. In certain embodiments, the plant cells secrete the cannabinoid compound(s) into the production medium; and/or the cannabinoid compound(s) are removed from the production medium on an ongoing basis. In some embodiments, there is an additional step of (b) obtaining the production medium from the bioreactor at the conclusion of the incubation; e.g. for obtaining residual cannabinoids not recovered during the previous steps. In certain embodiments, the cells are C. sativa cells. In more specific embodiments, the C. sativa cells are trichome cells.

In still other embodiments, there is provided a bioreactor, comprising: plant cells, a production medium, and one or more cannabinoid compounds disposed in the production medium. In certain embodiments, the bioreactor is configured for removing the cannabinoid compound(s) from the production medium on an ongoing basis. In various embodiments, the cannabinoid-producing cells are able to remain in the bioreactor during perfusion by virtue of their attachment to a 3D-matrix, physical protection from shear forces generated from currents in the medium, or a combination of both. In other embodiments, the bioreactor is configured for obtaining the production medium from the bioreactor at the conclusion of the incubation.

In yet other embodiments, there is provided a bioreactor, comprising: plant cells, a production medium, and one or more cannabinoid compounds disposed within the plant cells. In certain embodiments, the bioreactor is configured for harvesting the plant cells intact, or at least predominantly intact. In other embodiments, the bioreactor is configured for lysing the plant cells while inside the bioreactor at the conclusion of the incubation. Reference herein to cannabinoids compound(s) within a cell may refer, in various embodiments, to compounds predominantly inside they cytoplasm, predominantly within the cell wall, or a combination thereof. In more specific embodiments, more than 50% of the compounds are inside the cytoplasm; or, in other embodiments, more than 55%, more than 60%, more than 65%, more than 70%, more than 75%, more than 80%, more than 85%, more than 90%, or more than 95%. In other embodiments, more than 50% of the compounds are inside the cell wall; or, in other embodiments, more than 55%, more than 60%, more than 65%, more than 70%, more than 75%, more than 80%, more than 85%, more than 90%, or more than 95%.

In some embodiments of the described methods and compositions, the plant cells secrete more than 50% of the cannabinoid compound(s) produced thereby into the production medium. In other embodiments, the cells secrete more than 55%, more than 60%, more than 65%, more than 70%, more than 75%, more than 80%, more than 85%, more than 90%, more than 92%, more than 95%, more than 96%, more than 97%, more than 98%, or more than 99% of the cannabinoid compound(s) into the medium. Those skilled in the art will appreciate, in light of the present disclosure, that compounds secreted into growth medium can be obtained from the medium. In other embodiments, in the case of toxicity, plant cell growth and/or metabolism can be improved by ongoing removal of the compounds from the medium.

In still other embodiments, the plant cells retain more than 50% of the cannabinoid compound(s) produced thereby. Those skilled in the art will appreciate, in light of the present disclosure, that compounds retained in the cells can be obtained by harvesting the cells. In other embodiments, in the case of toxicity, plant cell growth and/or metabolism can be improved by modification of the compounds to reduce their toxicity, and/or by modification of the cells to confer protection from toxicity, e.g. as described herein.

Reference herein to removal of cannabinoids, or, in other embodiments, other metabolites, on an “ongoing” basis refers to removal of the compounds during cell incubation and/or during production of the compounds by the cells. “Ongoing” removal does not necessarily require continuous removal of the metabolites from the medium. In certain embodiments, removal of the metabolites is linked to perfusion of the bioreactor, at least on an intermittent basis. In other embodiments, perfusion keeps the metabolite concentration (which may be e.g. any of the cannabinoids mentioned herein, each of which represents a separate embodiment) to below 100 milligrams/liter (mg/L), 80 mg/L, 60 mg/L, 50 mg/L, 40 mg/L, 30 mg/L, 20 mg/L, 15 mg/L, 10 mg/L, 8 mg/L, 6 mg/L, 5 mg/L, 4 mg/L, 3 mg/L, 2 mg/L, 1.5 mg/L, 1 mg/L, 0.8 mg/L, 0.6 mg/L, 0.5 mg/L, 0.4 mg/L, 0.3 mg/L, 0.2 mg/L, 0.15 mg/L, 0.1 mg/L, 0.08 mg/L, 0.06 mg/L, 0.05 mg/L, 0.04 mg/L, 0.03 mg/L, 0.02 mg/L, 0.015 mg/L, or 0.01 mg/L. In further embodiments, the bioreactor comprises a sensor of cannabinoids (which may be, in more specific embodiments, THC, or in other embodiments CBD, or in other embodiments CBC, or in other embodiments CBN. The sensor, in further embodiments, controls the rate of perfusion and/or removal of cannabinoids from the bioreactor.

In certain embodiments, at an earlier stage of the method, the bioreactor is inoculated with the plant cells, which are, in some embodiments, introduced into the bioreactor while suspended in the production medium. In other embodiments, the plant cells are introduced into the bioreactor while disposed in a different liquid medium. In more specific embodiments, the bioreactor contains the described production medium.

In certain embodiments, the plant cells are incubated in the production medium for at least 3 hours, or, in other embodiments, for at least 4, 5, 6, 7, 8, 9, 10, 11, 12, 13, 14, 15, 16, 18, 20, 22, 24, 30, 36, 42, 48, 55, 60, 70, 80, 90, 100, 120, 140, 160, 180, or 200 hours. In other embodiments, the plant cells are incubated in the production medium for 3-50 hours, or, in other embodiments, for 3-100, 3-80, 3-60, 3-40, 3-30, 3-20, 3-15, 3-12, 3-10, 4-100, 4-80, 4-60, 4-50, 4-40, 4-30, 4-20, 4-15, 4-12, 4-10, 5-100, 5-80, 5-60, 5-50, 5-40, 5-30, 5-20, 5-15, 5-12, 5-10, 7-100, 7-80, 7-60, 7-40, 7-30, 7-20, 7-15, 7-12, 7-10, 10-100, 10-80, 10-60, 10-50, 10-40, 10-30, 10-20, 20-100, 20-80, 20-60, 20-50, 20-40, or 20-30 hours.

Alternatively or in addition, the described production medium comprises an inducing agent. In certain embodiments, the inducing agent induces a transcriptional activator of an inducible promoter for one or more genes that encode(s) a cannabinoid synthetic enzyme(s) (an enzyme that facilitates a step in a cannabinoid synthesis pathway) and/or a cannabinoid secretion enzyme(s) (an enzyme that facilitates secretion of a cannabinoid from a cell).

Except where indicated otherwise, reference herein to a suspension of cells encompasses both single-cell suspensions and suspensions of clumps of cells. In certain embodiments, clumps or single-cell suspensions may be introducing into, and expanded on, a growth apparatus including a 3D substrate, a 2D substrate, or no solid phase growth substrate. The embodiments may be freely combined.

Except where indicated otherwise, reference herein to plant cells synthesizing one or more cannabinoids encompasses instances where the cannabinoid(s) is either synthesized inside the plant cell from one or more relatively ubiquitous building blocks, e.g. hexanoic acid; or, in other embodiments, where the cell synthesizes the cannabinoid(s) from a more specific precursor compound, e.g. olivetolic acid. In either case, in various embodiments, the precursor may be naturally present in the plant cells and/or provided exogenously, for example supplied to the plant cell from the production medium.

In some embodiments, there is a provided a method of producing one or more cannabinoid compounds, comprising the steps of: (a) incubating plant cells in a bioreactor, under conditions fostering expansion of the plant cells; and (b) incubating plant cells in a production medium, inside a bioreactor, under conditions fostering secretion of cannabinoid compound(s) by the plant cells into the production medium, wherein the cannabinoid compound(s) are removed from the production medium on an ongoing basis; thereby producing cannabinoid compound(s). Optionally, an additional step of (c) obtaining the production medium from the bioreactor is performed e.g. for obtaining residual cannabinoids not recovered during the previous steps. In certain embodiments, at an earlier stage of the method, the bioreactor is inoculated with the plant cells, which are, in some embodiments, introduced into the bioreactor while suspended in the production medium. In other embodiments, the plant cells are introduced into the bioreactor while disposed in a different liquid medium. In more specific embodiments, the bioreactor contains the described production medium. In certain embodiments, the plant cells both synthesize and secrete the cannabinoids, while inside the bioreactor.

In more specific embodiments of the herein-described methods, the expansion phase is performed for 4-10 days, or, in other embodiments, 4-8, 4-7, 4-6, 3-8, 3-7, 3-6, 2-8, 2-6, 3-5, 4-20, 4-15, 4-12, 3-20, 3-15, 3-12, or 3-10 days.

In certain embodiments, at an earlier stage of the method, the bioreactor is inoculated with the plant cells, which are, in some embodiments, introduced into the bioreactor while suspended in the expansion medium. In other embodiments, the plant cells are introduced into the bioreactor while disposed in a different liquid medium. In more specific embodiments, the bioreactor contains the described expansion medium.

In other embodiments, the plant cells are incubated in the expansion medium for at least 3 population doublings; or, in other embodiments, at least 4, at least 5, at least 6, at least 8, at least 10, at least 12, at least 15, at least 17, or at least 20 population doublings. In still other embodiments, the plant cells are incubated in the expansion medium for at least 3 hours, or, in other embodiments, for at least 4, 5, 6, 7, 8, 9, 10, 11, 12, 13, 14, 15, 16, 18, 20, 22, 24, 30, 36, 42, 48, 55, 60, 70, 80, 90, 100, 120, 140, 160, 180, or 200 hours.

Alternatively or in addition, the plant cells are incubated in the production medium for at least 3 hours, or, in other embodiments, for at least 4, 5, 6, 7, 8, 9, 10, 11, 12, 13, 14, 15, 16, 18, 20, 22, 24, 30, 36, 42, 48, 55, or 60 hours. In other embodiments, the plant cells are incubated in the production medium for 3-50 hours, or, in other embodiments, for 3-100, 3-80, 3-60, 3-40, 3-30, 3-20, 3-15, 3-12, 3-10, 4-100, 4-80, 4-60, 4-50, 4-40, 4-30, 4-20, 4-15, 4-12, 4-10, 5-100, 5-80, 5-60, 5-50, 5-40, 5-30, 5-20, 5-15, 5-12, 5-10, 7-100, 7-80, 7-60, 7-40, 7-30, 7-20, 7-15, 7-12, 7-10, 10-100, 10-80, 10-60, 10-50, 10-40, 10-30, 10-20, 20-100, 20-80, 20-60, 20-50, 20-40, or 20-30 hours.

In more specific embodiments, conditions fostering expansion of the plant cells may comprise an expansion medium in which the cells can reach a peak population doubling time (PDT) of 10 days or less, 8 days or less, 7 days or less, 6 days or less, 5 days or less, 4 days or less, 3 days or less, 2 days or less, or 18 hours or less; or, in other embodiments, 2-10 days, 2-8 days, 2-7 days, 2-6 days, 2-5 days, 2-4 days, 3-10 days, 3-8 days, 3-7 days, 3-6 days, 3-5 days, or 3-4 days. In certain embodiments, the conditions fostering expansion of the plant cells elicit a relatively low level of synthesis of cannabinoids. In other embodiments, the level of synthesis of THC, or in other embodiments CBD, is at least 20-fold, at least 15-fold, at least 12-fold, at least 10-fold, at least 8-fold, at least 6-fold, at least 5-fold, at least 4-fold, at least 3-fold, or at least 2-fold lower than the cells' synthesis of THC, or in other embodiments CBD, in the described conditions fostering secretion of cannabinoid compound(s).

In certain embodiments, the PDT of the plant cells in the production medium is within 2-fold of the PDT in the expansion medium. In other embodiments, the 2 PDT's are within 20% of each other. In still other embodiments, the PDT in the production medium is at least 2-fold lower than the PDT in the expansion medium; or, in other embodiments, at least 3-fold, at least 4-fold, at least 5-fold, at least 6-fold, at least 8-fold, at least 10-fold, at least 12-fold, at least 15-fold, or at least 20-fold lower; or, in still other embodiments, 2-20 fold, 3-20 fold, 4-20 fold, 5-20 fold, 2-10 fold, 3-10 fold, 4-10 fold, or 5-10 fold lower than the PDT in the expansion medium. In certain embodiments, the lower PDT in the production medium is due to the toxicity of the cannabinoid(s) produced therein.

In other embodiments, the described expansion medium differs from the production medium, for example, by lacking one or more elicitors that are present in the described production medium. The term elicitor(s), except where indicated otherwise, refers herein to an elicitor of secondary metabolites in plants. In certain embodiments, the elicitor induces expression of THC, or in other embodiments CBD, or in other embodiments CBC, or in other embodiments CBN, in C. sativa cells. The induction (typically assessed at the maximum tolerated concentration of the elicitor) may be at least 2-fold, at least 3 fold, at least 5-fold, at least 7-fold, at least 10-fold, at least 15-fold, at least 20-fold, at least 30-fold, at least 50-fold, 2-50 fold, 3-50 fold, 5-50 fold, 7-50 fold, 10-50 fold, 20-50 fold, 2-20 fold, 3-20 fold, 5-20 fold, 7-20 fold, 10-20 fold, or 10-15 fold.

In certain embodiments, the elicitor is selected from jasmonic acid; methyl jasmonate; pectin; abscisic acid; salicylic acid; a heavy metal, non-limiting examples of which are copper, vanadium (e.g. vanadate, sodium orthovanadate, or vanadyl sulfate [VOSO₄]), and cadmium (e.g. CdCl₃ and CdCl₂); and ethylene. In other embodiments, the elicitor is selected from cannabis pectin extract, hydrolyzed cannabis pectin, sodium alginate, AgNO₃, CoCl₂, NiSO₄. In still other embodiments elicitor is selected from chitosan, chitin, and elicitin. Those skilled in the art will appreciate, in light of the present disclosure, that reference herein to elicitors includes derivatives thereof that exhibit the same biological activity. Elicitors are known in the art, and are described, inter alia, in Javier Lidoy Logroño (In vitro cell culture of Cannabis sativa for the production of cannabinoids. Bacherlor's thesis for Universitat Autònoma de Barcelona), Flores-Sanchez I J et al., Rao & Ravishankar, Ruffoni, B et al., and Vasconsuelo and Boland (2007).

Alternatively or in addition, the described production medium comprises an inducing agent. In certain embodiments, the inducing agent induces a transcriptional activator of an inducible promoter for one or more genes that encode(s) a cannabinoid synthetic enzyme(s) and/or a cannabinoid secretion enzyme(s).

In still other embodiments, UV light exposure acts as an elicitor of secondary metabolite production. In more specific embodiments the UV light may have a wavelength of between 300-400 nanometers (nm); or, in other embodiments between 280-315, 280-300, 290-315, 300-350, 350-400, 300-320, 320-340, 340-360, 360-380, or 380-400 nm.

The plant cells utilized in the described methods and compositions contain, in some embodiments, one or more genes that encode(s) a cannabinoid synthetic enzyme(s). Alternatively or in addition, the plant cells contain one or more gene that encode(s) a cannabinoid secretion enzyme(s). In certain embodiments, the genes are endogenous genes. In other embodiments, the genes are exogenous genes. In still other embodiments, the plant cells contain both endogenous and exogenous genes encoding cannabinoid synthetic enzyme(s) and/or cannabinoid secretion enzyme(s).

In yet other embodiments, the one or more genes that encode(s) a cannabinoid synthetic enzyme(s) and/or a cannabinoid secretion enzyme(s) is/or operably linked to an inducible promoter. Each possibility represents a separate embodiment.

The term “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters which initiate transcription only in certain tissues are referred to as “tissue-specific.”

The described inducible promoter is, in some embodiments, a Cannabis sativa MYB transcription factor-inducible promoter. In other embodiments, the promoter comprises a binding site for a transcriptional activator. Other non-limiting examples are those found in promoters from the ACEI system, which responds to copper; the In2 gene promoter from maize, which responds to benzenesulfonamide herbicide safeners; Tet repressor from Tn10; and the inducible promoter from a steroid hormone gene, the transcriptional activity of which may be induced by a glucocorticosteroid hormone (Schena et al. (1991) Proc. Natl. Acad. Sci. USA 88:0421). In further embodiments, the described production medium comprises an inducer of the transcriptional activator. Solely for exemplification, ethylene is an example of an inducer, in this case of Ethylene Response Factor (ERF) proteins (Zheng X et al., Ethylene response factor ERF11 activates BT4 transcription to regulate immunity to Pseudomonas syringae. Plant Physiol. 2019 Mar. 29); salicylic acid activates the Upstream Activation Sequence (UAS) of Figwort Mosaic Virus (Deb D and Dey N. Synthetic Salicylic acid inducible recombinant promoter for translational research. J Biotechnol. 2019 Mar. 14; 297:9-18. doi: 10.1016/j.jbiotec.2019.03.004. [Epub ahead of print]); and dexamethasone activates the transcription activator LhGR (Samalova M et al., Universal Methods for Transgene Induction Using the Dexamethasone-Inducible Transcription Activation System pOp6/LhGR in Arabidopsis and Other Plant Species. Curr Protoc Plant Biol. 2019 Mar. 12:e20089. doi: 10.1002/cppb.20089. [Epub ahead of print]). Those skilled in the art will appreciate, in light of the present disclosure, that neither the particular inducible promoter nor the particular transcriptional activator is critical to utilizing the described methods and compositions.

In other embodiments, the described plant cell comprises an expression vector containing one or more genes, which may be, in certain embodiments, any of the genes mentioned herein. An “expression vector” is a polynucleotide capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette”. In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides.

Alternatively or in addition, the described production medium comprises an inducing agent. In certain embodiments, the inducing agent induces a transcriptional activator of an inducible promoter for one or more genes that encode(s) a cannabinoid synthetic enzyme(s) and/or a cannabinoid secretion enzyme(s).

In still other embodiments, the described methods further comprise monitoring and maintaining homeostasis of (or controlling) at least one of pH, temperature, and levels of dissolved oxygen, glucose, lactate, lactate dehydrogenase, NH₃, and glutamate, each of which represents a separate embodiment. The monitoring and controlling of the described parameters may take place during incubation in the expansion medium and/or the production medium, each of which may be freely combined with the mentioned parameters.

Except where indicated otherwise, reference herein to an enzyme includes all its isoforms functional fragments thereof, and mimetics thereof. Such reference also includes homologues from a variety of species, provided that the protein acts on the substrate in a similar fashion to the described enzyme.

Cannabinoid Compounds

The terms cannabinoids and cannabinoid compounds may refer, in some embodiments, to various cannabinoids known in the art. In certain embodiments, the term encompasses THC. In other embodiments, the term encompasses CBD. In still other embodiments, the term encompasses both THC and CBD. In yet other embodiments, the term encompasses THC, CBD, CBC, and CBG. Reference in this paragraph to specific compounds does not preclude, in some embodiments, the production and/or presence of additional cannabinoids. In other embodiments, the term encompasses tetrahydrocannabiphorol (Citti C, et al). In still other embodiments, cannabinoids refers specifically to any cannabinoid compound known in the art, each of which represents a separate embodiment. Cannabinoids are known in the art, and are described, for example, in Aizpurua-Olaizola O et al.

Without wishing to be bound by theory, THC acts through CB1 and CB2 receptors of the endocannabinoid system. It is a partial agonist of both receptors; however, it exhibits higher affinity for the CB1 receptor, which is believed to be responsible for the psychoactive effect of THC, but also for its analgesic and antispastic action. CB1 receptors are mainly located in the central nervous system; however, they are also found in the cells of the immune system, digestive system, reproductive system, heart, lungs, adrenal glands and bladder, which explains its wide spectrum of action (Andre et al. 2016). On the other hand, the CB2 receptor is responsible for modulating the immune system by regulating cytokine activity. Its location overlaps with the peripheral nervous system and immune system, which may be attributed to its analgesic and anti-inflammatory action (Burstein 2015).

CBD is characterized by antipsychotic, anti-anxiety, anti-inflammatory and antioxidant properties and has no toxic effect on human health in doses from 10 mg up to even 700 mg (Zuardi et al. 2006; Pryce et al. 2015). CBD can limit or alleviate the psychoactive effect of THC (Englund et al. 2013). Studies on cannabinoid activity also suggest its antineoplastic action (Velasco et al. 2012; Haustein et al. 2014).

In addition to the THC and CBD, other minor cannabinoids, such as CBC and CBN (cannabinol) have potential therapeutic applications. CBC inhibits the reuptake of anandamide—an endogenous ligand of CB receptors (De Petrocellis et al. 2011). CBN has twofold lower affinity for the CB1 receptor and threefold higher affinity for the CB3 receptor than THC, consistent with a positive effect on the immune system (Andre et al. 2016).

Basal Media

Those skilled in the art will appreciate, in light of the present disclosure, that the production media and expansion media in the described methods and compositions may independently be based on a variety of basal media. In certain embodiments, the expansion medium or production medium contains one or more major salts (macronutrients), non-limiting examples of which are Ammonium nitrate (NH₄NO₃), Calcium chloride (CaCl₂.2H₂O), Magnesium sulfate (MgSO₄.7H₂O), Monopotassium phosphate (KH₂PO₄), Potassium nitrate (KNO₃), and Potassium phosphate; one or more minor salts (micronutrients), non-limiting examples of which are Boric acid (H₃BO₃), Cobalt chloride (CoCl₂.6H₂O), Ferrous sulfate (FeSO₄.7H₂O), Manganese(II) sulfate (MnSO₄.4H₂O), Potassium iodide (KI), Sodium molybdate (Na₂MoO₄.2H₂O), Zinc sulfate (ZnSO₄.7H₂O), Ethylenediaminetetraacetic acid ferric sodium (NaFe-EDTA), EDTA, and Copper sulfate (CuSO₄.5H₂O); and one or more vitamins/organic substances, non-limiting examples of which are Myo-Inositol, Nicotinic Acid, Pyridoxine, Thiamine, Glycine. Optional additions include Edamin™ or tryptone (optional), Indole Acetic Acid (optional), Kinetin, and a carbohydrate. One or more of each of the aforementioned major salts, minor salts and vitamins/organic substances may be freely combined.

A non-limiting example of media useful in the described methods and compositions is Murashige-Skoog (MS) medium, which contains the following components (concentrations in grams/liter [g/L] in parentheses: Ammonium nitrate (1650), Calcium chloride (332), Magnesium sulfate (180), Potassium nitrate (1900), Potassium phosphate monobasic (170), Boric acid (6.2), Cobalt chloride hexahydrate (0.025), Copper sulfate pentahydrate (0.025), EDTA disodium salt dihydrate (37.3), Ferrous sulfate heptahydrate (27.8), Manganese sulfate monohydrate (16.9), and Molybdic acid (sodium salt) 0.213. Optionally, MS medium may be supplemented with Edamin™ or tryptone 1 g/l (optional), Indole Acetic Acid 1-30 mg/l (optional), and/or Kinetin 0.04-10 mg/l (optional).

Yet another embodiment of a useful medium is B5 medium, containing the following ingredients (in mg/L): Ammonium sulphate (134), Calcium chloride (113.2), Magnesium sulphate (122.1), Potassium nitrate (2500), Sodium phosphate monobasic (130.4) Boric acid (3.0), Cobalt chloride hexahydrate (0.025), Copper sulphate pentahydrate (0.025), EDTA disodium salt dihydrate (37.3), Ferrous sulphate heptahydrate (27.8), Manganese sulphate monohydrate (10.0), Molybdic acid (sodium salt) 0.213, Potassium Iodide Zinc (0.75), sulphate heptahydrate (2), myo-Inositol (optional; 50-100), Nicotinic acid (free acid) (optional; 1.0), Pyridoxine HCl (1.0), and Thiamine hydrochloride (optional; 10), casein hydrolysate (optional; 1000) and supplemented with 3% sucrose.

Other, non-limiting embodiments of useful media are White's medium (White 1939) and woody plant medium (Lloyd and McCown).

Those skilled in the art will appreciated, in light of the present disclosure, that the media described herein are solely exemplary, and other suitable media may be used; that many of the components may be omitted or replaced; and that the amounts of the components may be modified without adversely affecting the growth-supporting properties of the medium. Typically, useful media will have vitamin supplementation (e.g., see Gamborg O L et al.) and sources of nitrogen, phosphorus, and potassium. Known media may be combined in various components. All of this is within the ability of the skilled person.

In other embodiments, the medium, e.g. the production medium, further comprises one or more phytohormones, non-limiting examples of which are auxins (non-limiting examples of which are Indole-3-acetic acid [IAA], 4-Chloroindole-3-acetic acid [4-CI-IAA], 2-phenylacetic acid [PAA], Indole-3-butyric acid [IBA], Indole-3-propionic acid [IPA], 1-naphthaleneacetic acid [NAA], and 2,4-dichlorophenoxyacetic acid [2,4-D]), cytokinins (non-limiting examples of which are adenine-type cytokinins [e.g. Kinetin, Zeatin, and 6-benzylaminopurine (also known as BA or benzyl adenine)] and phenylurea-type cytokinins (e.g. diphenylurea and thidiazuron [TDZ] [Aina et al.]), gibberellic acid (GA₃), brassinosteroids, ethylene, abscisic acid, salicylic acid and jasmonic acid, each one of which represents a separate embodiment. In still other embodiments, the medium further comprises one or more elicitors, non-limiting examples of which are mentioned herein.

Alternatively or in addition, the medium, e.g. the production medium, further comprises one or more chemicals, metal ions, and/or catalysts that detoxify hydrogen peroxide (H₂O₂), non-limiting examples of which are manganese dioxide (MnO₂), permanganate ion (MnO₄), silver ion (Ag+), iron oxide, (Fe₂O₃), lead dioxide (PbO₂), cupric oxide (CuO), Hafnium(IV) oxide (HfO₂), cerium dioxide (CeO₂), gadolinium trioxide (Gd₂O₃), Sodium Phosphate, Tribasic (NaPO₄), iodide ions, manganese metal, and iron(III) Chloride Solution (FeCl₃).

In still other embodiments the described production medium comprises an elicitor, which may be, e.g. a type of elicitor described herein.

In further embodiments, the described medium is supplemented with a carbohydrate, non-limiting examples of which are sugars. When present, the sugar (e.g. sucrose, glucose, or fructose), is present, in further embodiments, at a concentration of 5-30 g/L, or, in other embodiments, 5-50, 5-40, 5-20, 5-15, 5-10, 3-50, 3-40, 3-30, 3-20, 3-15, 3-10, 3-8, 7-50, 7-40, 7-30, 7-20, 7-15, 7-10, 10-50, 10-40, 10-30, 10-20, or 10-15 g/L.

It will be appreciated that additional components may be added to the culture medium. Such components may be antibiotics, antimycotics, albumin, amino acids, and other components known to the art for the culture of cells.

Plant Cells

In certain embodiments, the cells that are subjected to the described methods are adherent plant cells, which may be, in more specific embodiments, a C. sativa cell. In other embodiments, the plant cell is from another plant. In more specific embodiments, the C. sativa cells are trichome cells. In more specific embodiments, trichomes are selected from leaf-trichomes, capitate-stalked trichomes, and capitate-sessile trichome, each of which represents a separate embodiment. In certain embodiments, the cells are isolated from one or more glandular trichomes.

Alternatively or in addition, the cells are subject to culturing steps on liquid, solid, or semi-solid media (for example, those detailed hereinbelow) prior to the herein-described bioreactor incubation steps.

In certain embodiments, the plant cells are expanded on a 2D scaffold (e.g. in a tissue culture apparatus), prior to the herein-described bioreactor incubation steps.

In some embodiments, C. sativa cells are subjected to callus culturing. In certain embodiments, the term callus culture/culturing refers to a culture of clumps of undifferentiated cells, e.g. parenchymal cells. In further embodiments, the clumps are actively dividing cells in the form of a non-organized tissue. Those skilled in the art will appreciate, in light of the present disclosure, that callus cultures can be derived from injury (wounding) of differentiated tissue culture (Pierik 1987). In still other embodiments, callus tissue, after it has been established, is maintained in an actively growing state by the transfer of fragments to a fresh medium at regular intervals, for example, 4-week intervals (Remotti and Loffler 1995). In certain embodiments, glutamine-free medium is used.

Another non-limiting example of callus culturing is described in Braemer and Paris (1987). Cell cultures are obtained from callus cultures of leaf explants and cultured in suspension culture in B5 medium supplemented with 0.5 mg/L Kinetin and 1 mg/L 2,4-dichiorophenoxyacetic acid, on rotary shaker at 120 rpm and 25° C. in total darkness. Subcultures are transferred to fresh medium every three weeks. Yet another exemplary protocol is described in Flores-Sanchez et al. (20 09). Cell cultures are obtained from callus cultures of leaves and cultured in suspension, in MS basal medium supplied with B5 vitamins, 1 mg/L 2,4-D and 1 mg/L Kinetin, on an orbital shaker at 110 rpm and under a light intensity of 1000-1700 lx. at 25° C. Subcultures are transferred to fresh medium every two weeks.

Additional, non-limiting protocols for callus induction are described in Mustafa et al. Mustafa also describes methods for producing suspension cultures from calli, and subculturing by either filtration, decanting or pipetting, or treatment with Pectinase, each of which represents a separate embodiment. Furthermore, Mustafa describes methods of subculturing, by suction through a filtrate, pipetting, or pouring, each of which represents a separate embodiment.

Additional non-limiting examples of callus culturing are described in Hussein. For callogenesis (callus induction), juvenile leaves are cut into pieces (“explants”) and cultured in modified B5 medium (Table 1) with 50 mg/l myo-inositol, 10 mg/l thiamine HCl, 1000 mg/l casein hydrolysate; supplemented with 3% sucrose; and solidified with 0.4% Gelrite®. 1-3 mg/L of combinations of 2,4-D, NAA, KIN and/or BA (specifically NAA+KIN, 2,4-D+BA, or NAA+BA) are added to stimulate callus induction. Callus cultures are subcultured every 20 days and again incubated under the aforementioned conditions. Optimal callus formation was seen with the combination of NAA+BA.

In certain embodiments, meristemoid formation is induced in callus cultures. Hussein describes a non-limiting, exemplary protocol. Calli are transferred onto solid modified B5 basal as described above with addition of 0.25, 0.5, 1, or 1.5 mg/L NAA; 10, 5, 3, or 1 mg/L BA; and 40 mg/L adenine hemisulfate salt (AS). Calli are subcultured at 20-day intervals under the conditions described in the previous paragraph. After 20 days, meristemoid-forming calli are cut into small pieces and subcultured on the same medium. The optimum combination for meristemoid induction was NAA/BA/AS.

In other embodiments, calli (e.g. with meristemoids) are subjected to shootlet induction. Hussein describes a non-limiting, exemplary protocol. Calli treated for meristemoid formation are used for shootlet induction after 5 months of growth. Calli are incubated on modified B5 medium supplemented with 0.25, 0.5, 1, or 1.5 mg/L cytokinins (6-benzylaminopurine, Zeatin, Kinetin, or thidiazuron); and/or 0.25, 0.5, 1, 1.5, or 3 mg/L gibberellic acid. After 1 month, shootlet-forming calli are cut into small pieces and subcultured on the same medium. 0.25 mg/l TDZ+3 mg/l GA₃ was optimum for shootlet formation.

In still other embodiments, shootlets are treated to induce root formation. Hussein describes a non-limiting, exemplary protocol. Single shootlets showing normal development are separated and cultivated in beakers containing solidified B5 medium supplemented with various concentrations (0.5, 1.0 or 1.5 mg/1) of NAA, IBA or IAA to induce root formation. Shoot cultures are incubated for 1 week in darkness at 20° C., then transferred into a lighted incubated and grown for 4 weeks as described above. 1.5 mg/l IAA was optimum for root formation.

In still other embodiments, callus cultures are subject to subsequent shake flask suspension culturing. Hussein describes a non-limiting, exemplary protocol. Cell clusters may be transferred to modified B5 liquid medium, supplemented with various concentrations of BA and TDZ, alone or in combination with GA3 (Table 2), and cultured for 2-5 weeks. Optimum growth was found in 1-1.5 mg/L TDZ+1.5 mg/L GA₃. Other, non-limiting exemplary methods are described in Mustafa et al. and the references cited therein. In certain embodiments, one or more auxins but no cytokinins are present in the medium.

In certain embodiments, batch shake flask suspension culturing of plant cells is performed prior to bioreactor culturing under perfusion. In other embodiments, suspension culturing is performed in a bioreactor from the beginning.

In still other embodiments, callus cultures are subject to hairy root induction. Hussein describes a non-limiting, exemplary protocol. Callus cultures may be incubated on B5 medium modified with 50 mg/l myo-inositol, 10 mg/l thiamine HCl, and 1000 mg/l casein hydrolysate; supplemented with 3% sucrose and various concentrations (1.5, 2.5 and 4 mg/L) of NAA, IBA and IAA; and solidified with 0.4% Gelrite®. Optimum hairy root formation was seen with 4 mg/L NAA.

Emerging hairy root cultures are isolated from callus cultures and subsequently transferred to solid B5 medium with 4 mg/l NAA, in the dark, at 25° C., and are subcultured each 30 days. Subsequently, established adventitious root cultures may be cut transversely and placed in half-solid B5 medium with 0.25, 0.5 or 1.5 mg/l of NAA, IBA or IAA in the dark at 25° C. A liquid culture is obtained by incubating the root tips in B5 medium supplemented with 4 mg/L NAA, in the dark at 25° C. on a rotary shaker, subculturing each 30 days.

In certain embodiments, batch shake flask suspension culturing of hairy root structures is performed prior to bioreactor culturing under perfusion. In other embodiments, suspension culturing is performed in a bioreactor from the beginning.

In yet other embodiments, callus cultures are subject to subsequent trichome induction. Hussein describes a non-limiting, exemplary protocol. Calli cultures are transferred onto solid B5 basal medium supplemented with 0.1, 0.3, 0.5, or 1.0 mg/L TDZ and/or 3 mg/L GA₃, under the same culture conditions as callus induction. Optimum trichome production was found with 0.5-1.0 mg/L TDZ+3 mg/L GA₃,

In certain embodiments, batch shake flask suspension culturing of trichomes is performed prior to bioreactor culturing under perfusion. In other embodiments, suspension culturing is performed in a bioreactor from the beginning.

In certain embodiments, the trichome cells are isolated by isolating the trichome structure (which may be performed by dissection, or any other suitable procedure) and incubating the trichome cells in a nutrient medium; which may be, in non-limiting embodiments the described expansion medium or production medium. In more specific embodiments, the trichome may be subject to disassociation, e.g. by mechanical agitation and/or incubation with a proteolytic enzyme. Following disassociation, in some embodiments, the cells exist in small clumps, e.g. in clumps of 2-20 cells, or in other embodiments 2-30, 2-40, 2-50, 2-15, 2-10, 3-10, 3-15, 3-20, 3-30, 3-40, 3-50, 5-10, 5-15, 5-20, 5-30, 5-40, or 5-50 cells. For example, at least 50%, at least 60%, at least 70%, at least 80%, at least 90%, at least 95%, at least 97%, at least 98%, at least 99%, or at least 99% of the cells may exist in small clumps. In certain embodiments, the plant cells exist as clumps in the bioreactor.

In other embodiments, the trichome cells exist, after disassociation, as single cells. For example, at least 50%, at least 60%, at least 70%, at least 80%, at least 90%, at least 95%, at least 97%, at least 98%, at least 99%, or at least 99% of the cells may be single cells.

Methods of isolating trichome cells are known in the art. An exemplary, non-limiting protocol is found in Zhang and Oppenheimer. In some embodiments, C. sativa leaves are optionally fixed (using a reversable fixative), followed by washing to remove the fixative; incubated with a chelator of divalent cations (e.g. EGTA or EDTA); gently abraded; and washed and resuspended in optionally isotonic buffer solution. In certain embodiments, the isolated trichomes are subjected to filtration through a cell strainer. Typically, useful cell strainers have pores of 70-100 microns.

TABLE 1
Modified B5 medium.

| Ingredients | mg/l |
|---|---|
| CoCl2•6H2O | 0.025 |
| CuSO4•5H2O | 0.025 |
| FeNaEDTA | 36.70 |
| H3BO3 | 3.00 |
| KI | 0.75 |
| MnSO4•H2O | 10.00 |
| NaMoO4•2H2O | 0.25 |
| ZnSO4•7H2O | 2.00 |
| CaCl | 113.23 |
| KNO3 | 2500.00 |
| MgSO4 | 121.56 |
| NaH2PO4 | 130.44 |
| (NH4)2SO4 | 134.00 |
| i-Inositol | 100.00 |
| nicotinic acid | 1.00 |
| Pyridoxine HCl | 1.00 |
| Thiamine HCl | 10.00 |
| Sucrose | 30000.00 |

TABLE 2
Modified B5 liquid medium.

| | | |
|---|---|---|
| Medium | Cytokinin | Auxin |

Liquid media

| | | |
|---|---|---|
| 1 | 0.5 mg/L BA | — |
| 2 | 1.0 mg/L BA | — |
| 3 | 1.5 mg/L BA | — |
| 4 | 0.5 mg/L TDZ | — |
| 5 | 1.0 mg/L TDZ | — |
| 6 | 1.5 mg/L TDZ | — |
| 7 | — | 1.5 mg/L GA₃ |
| 8 | 0.5 mg/L BA | 1.5 mg/L GA₃ |
| 9 | 1.0 mg/L BA | 1.5 mg/L GA₃ |
| 10 | 1.5 mg/L BA | 1.5 mg/L GA₃ |
| 11 | 0.5 mg/L TDZ | 1.5 mg/L GA₃ |
| 12 | 1.0 mg/L TDZ | 1.5 mg/L GA₃ |
| 13 | 1.5 mg/L TDZ | 1.5 mg/L GA₃ |

More generally, in certain embodiments, callus culturing is performed by mincing plant tissues (which may be, in more specific embodiments, leaves, or, in other embodiments, female flowers containing trichomes), surface-sterilizing the pieces, and culturing them a gelled nutrient medium, or in other embodiments in suspension in nutrient medium with stirring. In still other embodiments initial culturing is in gelled medium, followed by suspension culturing in liquid medium. In other embodiments, the tissue fragments are cultured in the presence of a 3D adhesion substrate, or, in still other embodiments, a 2D adhesion substrate. Optionally, if required, the cell clumps are passaged to fresh medium every 1-4 weeks. Alternatively or in addition, the medium comprises one or more additives that stimulates callus induction, non-limiting examples of which are the cytokinin Kinetin (e.g. at 0.5 mg/L), in some embodiments in combination with the auxin 2,4-dichiorophenoxyacetic acid (e.g. at 1 mg/L), NAA, KIN and BA. In certain embodiments, specific ranges of auxin to cytokinin ratios are used to favor growth and maintenance of an unorganized growing and dividing mass of callus cells, as is known to those skilled in the art. Those skilled in the art will appreciate, in light of the present disclosure, that the particular method of inducing callus induction is not critical to carrying out the described methods.

Alternatively or in addition, a shaker (which may be, in more specific embodiments, an orbital shaker) is used to mix the culture. In certain embodiments, the shaker is rotated at 50-200 rpm, or, in other embodiments, 50-150, 50-120, 50-110, 50-100, 60-200, 60-150, 60-120, 60-110, 80-200, 80-150, 80-120, 80-110, 100-200, 100-150, 100-120, 110-200, 110-150, 110-130, or 150-200 rpm.

In still other embodiments, the culture, at least at certain stages, is incubated under light, which may be, in more specific embodiments, a light intensity of 500-3000 lux, or, in other embodiments, 500-2500, 500-2000, 500-1700, 1000-3000, 1000-2500, 1000-2000, or 1000-1700 lux. In more specific embodiments, callus induction and/or callus incubation is performed in the light.

In still other embodiments, the culture, at least at certain stages, is incubated in the dark.

The temperature of the culture is, in certain embodiments, 20-30° C.; or, in other embodiments, 20-37, 20-35, 20-32, 22-37, 22-35, 22-32, 22-30, 25-37, 25-35, 25-32, 25-30, 21-29, 22-28, 23-27, or 24-26° C.

In yet other embodiments, plant parts and/or fragments thereof (e.g. flowers, or in other embodiments leaves) are incubated in nutrient medium, and cells that migrate out of the parts or fragments are used in subsequent culturing. In certain embodiments, the migrating cells are dedifferentiated cells with relatively high proliferation potential.

All the described embodiments of various parameters of culturing (e.g. callus induction and culturing, shake flask culturing, hairy root induction and culturing, etc.) may be freely combined.

In certain embodiments, further steps of purification or enrichment may be performed. Such methods include, but are not limited to, cell sorting using markers for a particular plant cell, a non-limiting example of which is a C. sativa trichome cell. Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

Cell Stocks

In still other embodiments, the described expanded plant cells are used to create a chilled cell stock. In some embodiments, the stock contains frozen trichomes, or in other embodiments cells derived therefrom. In other embodiments, the stock contains frozen calli, or in other embodiments cells derived therefrom. The stock may be used to directly inoculate a bioreactor, or, in other embodiments, for further culturing (e.g. trichome production from a stock such as calli cells), followed by bioreactor inoculation.

In certain embodiments, one or more excipients or carriers are present together with the plant cells, e.g., within a frozen stock of trichomes, calli, or other formulations of plant cells. Examples, without limitations, of carriers are propylene glycol and saline. In some embodiments, the pharmaceutical carrier is an aqueous solution of saline. In other embodiments, the composition further comprises an excipient compatible with maintaining cellular viability, e.g. at chilled temperatures. Non-limiting examples of such excipients are DMSO (dimethyl sulfoxide, for example at concentrations of 3%-10%) and non-reducing disaccharides (e.g. trehalose and sucrose, for example at concentrations of 100 mM-1.5 M). Other, non-limiting examples of cryoprotectants are penetrating cryoprotectants such as glycerol and 1,2-propanediol, and non-penetrating cryoprotectants such as polyvinyl pyrrolidone, fructose, and glucose. In further embodiments, the excipient is a cryoprotectant, or is a carrier protein. Alternatively or in addition, the composition is frozen.

In other embodiments, for injection, the cells are stored in an aqueous solution, e.g. in physiologically compatible buffers such as Hank's solution, Ringer's solution, or physiological salt buffer, optionally in combination with medium containing cryopreservation agents.

In more specific embodiments, the cell stock is used to seed bioreactors, or in other embodiments to seed a tissue culture apparatus, to produce numerous lots of cannabinoids. In certain embodiments, the stock is created from a preferred strain. In still other embodiments, the stock enables consistency between bioreactor batches.

Additional Method Steps and Characteristics

In other embodiments, the described expansion and/or production steps independently utilize microcarriers, which may, in certain embodiments, be inside a bioreactor. Microcarriers are known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.,) Superbeads® (commercially available from Flow Labs, Inc.,), and as DE-52 and DE-53 (commercially available from Whatman, Inc.). In certain embodiments, the microcarriers are packed inside a bioreactor. In other embodiments, any type of 3D adherent substrate is utilized.

In still other embodiments, a fixed-bed bioreactor is utilized. In certain embodiments, the fixed-bed bioreactor protects cells from shear forces, for example as described in Nagai et al.

In yet other embodiments, the described bioreactor is selected from a (a) mechanically agitated bioreactor (e.g. aeration-agitated bioreactors, rotating drum bioreactors, and spin filter bioreactors), (b) a pneumatically driven bioreactor (e.g. unstirred bubble bioreactors, bubble column bioreactors [BCBs], air-lift bioreactors) and (c) a non-agitated system (e.g. gaseous phase bioreactors, oxygen permeable membrane aerator bioreactors, and overlay aeration bioreactors). In still other embodiments, a stirred tank reactor, helical ribbon or double helical ribbon impeller bioreactor, rotating wall vessel bioreactor, a membrane bioreactor, a hollow-fiber system, a tubular membrane bioreactor, a silicone-tubing aerated bioreactor, a slug bubble bioreactor, a wave bioreactor, an orbitally shaken bioreactor, or a super spinner bioreactor (e.g. see Furusaki and Takeda, Takayama and Akita, Valdiani et al., and the references cited therein). Each type of bioreactor represents a separate embodiment.

In still other embodiments, the expansion and/or production steps independently utilize a 2D adherent substrate.

In certain embodiments, the expansion step is performed on a 2D substrate, and the production step is performed on a 3D substrate. In still other embodiments, at least a portion of the production step is performed on a 3D substrate. In further embodiments, the expansion step and a portion of the production step are performed on a 2D substrate, and the remainder of the production step is performed on a 3D substrate. In more specific embodiments, the last portion of the production step is performed on a 3D substrate, for at least 3 hours, or, in other embodiments, for at least 4, 5, 6, 7, 8, 9, 10, 11, 12, 13, 14, 15, 16, 18, 20, 22, 24, 30, 36, 42, 48, 55, or 60 hours.

In yet other embodiments, the expansion and/or production steps independently do not utilize a solid-phase growth substrate.

Bioreactors

In certain embodiments, the described methods, or certain steps thereof, are performed in a bioreactor. In some embodiments, the bioreactor comprises a container for holding medium and a 3D attachment (carrier) substrate disposed therein, and a control apparatus, for controlling pH, temperature, and oxygen levels and optionally other parameters. In more specific embodiments, the 3D substrate is in a packed bed configuration. Alternatively or in addition, the bioreactor contains ports for the inflow and outflow of fresh medium and gases. In certain embodiments, the expansion step is performed in a tissue culture apparatus, and the production step is performed in a bioreactor. In still other embodiments, at least a portion of the production step is performed in a bioreactor. In further embodiments, the expansion step and a portion of the production step are performed in a tissue culture apparatus, and the remainder of the production step is performed in a bioreactor. In more specific embodiments, the last portion of the production step is performed in the bioreactor, for at least 3 hours, or, in other embodiments, for at least 4, 5, 6, 7, 8, 9, 10, 11, 12, 13, 14, 15, 16, 18, 20, 22, 24, 30, 36, 42, 48, 55, or 60 hours.

In certain embodiments, the aforementioned bioreactor is a packed-bed bioreactor. In some embodiments, the bioreactor comprises a container for holding medium, and a control apparatus, for controlling pH, temperature, and oxygen levels and optionally other parameters. In more specific embodiments, the bioreactor also contains a 3D substrate. Alternatively or in addition, the bioreactor contains ports for the inflow and outflow of fresh medium and gases.

In certain embodiments, the bioreactor is connected to an external medium reservoir (e.g. that is used to perfuse the bioreactor).

The term packed-bed bioreactor, except where indicated otherwise, refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during agitation at the standard agitation rate of the bioreactor. In certain embodiments, the definition does not exclude that the substrate may be lifted at unusually fast agitation rates, for example greater than 200 rpm.

Examples of bioreactors include, but are not limited to, a continuous stirred tank bioreactor, a CelliGen® bioreactor system (New Brunswick Scientific (NBS) and a BIOFLO 310 bioreactor system (New Brunswick Scientific (NBS).

In certain embodiments, a bioreactor is capable, in certain embodiments, of expansion of cells on a 3D substrate under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Additional bioreactors that may be used, in some embodiments, are stationary-bed bioreactors; and air-lift bioreactors, where air is typically fed into the bottom of a central draught tube flowing up while forming bubbles, and disengaging exhaust gas at the top of the column. Additional possibilities are cell-seeding perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference.

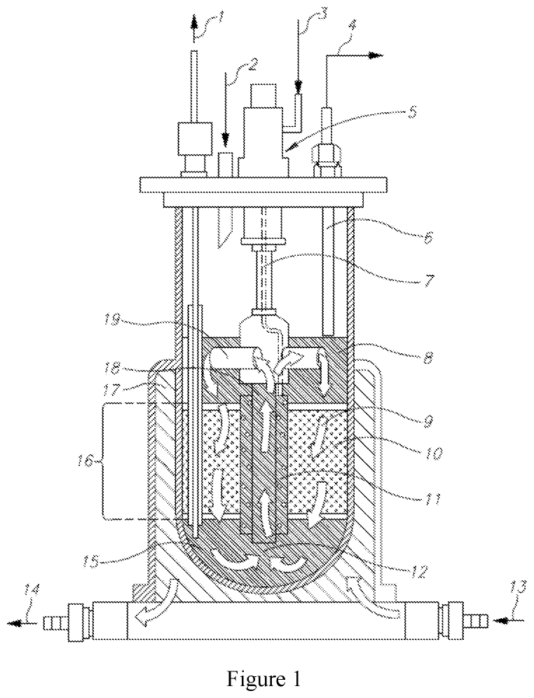

Another exemplary bioreactor, the CelliGen 310 Bioreactor, is depicted in FIG. 1. In the depicted embodiment, A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow initial stirring rate is used to promote cell attachment, then the stirring rate is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains a 3D substrate. In certain embodiments, the 3D substrate is in the form of carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex®, GE Healthcare), collagen-based (Cultispher®, Percell Biolytica), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion.

Alternatively or in addition, the carriers comprise a fibrous material, optionally an adherent, fibrous material, which may be, in more specific embodiments, a woven fibrous matrix, a non-woven fibrous matrix, or either. Non-limiting examples of fibrous carriers are New Brunswick Scientific Fibracel® carriers, available commercially from of Eppendorf AG, Germany, and made of polyester and polypropylene; and BioNOC II carriers, available commercially from CESCO BioProducts (Atlanta, Ga.) and made of PET (polyethylene terephthalate). In certain embodiments, the referred-to fibrous matrix comprises a polyester, a polypropylene, a polyalkylene, a polyfluorochloroethylene, a polyvinyl chloride, a polystyrene, or a polysulfone. In more particular embodiments, the fibrous matrix is selected from a polyester and a polypropylene.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus, which may be, in more specific embodiments, a fibrous matrix; a non-woven fibrous matrix; non-woven fibrous matrix comprising polyester; or a non-woven fibrous matrix comprising at least about 50% polyester. In more specific embodiments, the matrix may be similar to the CelliGen™ Plug Flow bioreactor which is, in certain embodiments, packed with Fibra-cel® (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the stirring speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700, between 450-650, between 475-625, between 500-600, or between 525-575 mg\liter. In yet other embodiments, at the end of the culture process, the carriers are removed from the packed bed and, in some embodiments, washed with isotonic buffer, and the cells are processed or removed from the carriers by agitation and/or enzymatic digestion.

In certain embodiments, the bioreactor is seeded at a concentration of between 10,000-2,000,000 cells/mL of medium, or, in various embodiments 20,000-2,000,000, 30,000-1,500,000, 40,000-1,400,000, 50,000-1,300,000, 60,000-1,200,000, 70,000-1,100,000, 80,000-1,000,000, 80,000-900,000, 80,000-800,000, 80,000-700,000, 80,000-600,000, 80,000-500,000, 80,000-400,000, 90,000-300,000, 90,000-250,000, 90,000-200,000, 100,000-200,000, 110,000-1,900,000, 120,000-1,800,000, 130,000-1,700,000, or 140,000-1,600,000 cells/mL.

In still other embodiments, between 1-20×10⁶ cells per gram (gr) of carrier (substrate) are seeded, or in other embodiments 1.5-20×10⁶ cells/gr carrier, or in other embodiments 1.5-18×10⁶ cells/gr carrier, or in other embodiments 1.8-18×10⁶ cells/gr carrier, or in other embodiments 2-18×10⁶ cells/gr carrier, or in other embodiments 3-18×10⁶ cells/gr carrier, or in other embodiments 2.5-15×10⁶ cells/gr carrier, or in other embodiments 3-15×10⁶ cells/gr carrier, or in other embodiments 3-14×10⁶ cells/gr carrier, or in other embodiments 3-12×10⁶ cells/gr carrier, or in other embodiments 3.5-12×10⁶ cells/gr carrier, or in other embodiments 3-10×10⁶ cells/gr carrier, or in other embodiments 3-9×10⁶ cells/gr carrier, or in other embodiments 4-9×10⁶ cells/gr carrier, or in other embodiments 4-8×10⁶ cells/gr carrier, or in other embodiments 4-7×10⁶ cells/gr carrier, or in other embodiments 4.5-6.5×10⁶ cells/gr carrier.

Adherent Materials

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or either. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a polycarbonate, a cellulose acetate, a glass fiber, a ceramic particle, a poly-L-lactic acid, and an inert metal fiber. Other embodiments include Matrigel™, an extra-cellular matrix component (e.g., Fibronectin, Chondronectin, Laminin; or a fragment thereof), and a collagen. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polycarbonates, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen. In still other embodiments, the adherent material is coated with an extra-cellular matrix component (non-limiting examples of which are Fibronectin, Chondronectin, Laminin; or a fragment thereof). In still other embodiments, the adherent material is coated with an extra-cellular matrix component (non-limiting examples of which are Fibronectin, Chondronectin, Laminin; or a fragment thereof).

Alternatively or in addition, the adherent material is fibrous, which may be, in more specific embodiments, a woven fibrous matrix, a non-woven fibrous matrix, or either. In still other embodiments, the material exhibits a chemical structure such as charged surface groups, which allows cell adhesion, e.g. polyesters, polypropylenes, polyalkylenes, polyfluorochloroethylenes, polyvinyl chlorides, polystyrenes, polysulfones, cellulose acetates, and poly-L-lactic acids. In more particular embodiments, the material may be selected from a polyester and a polypropylene.

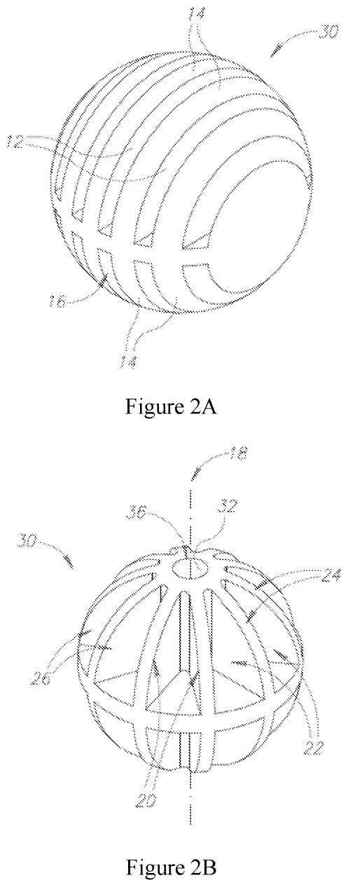

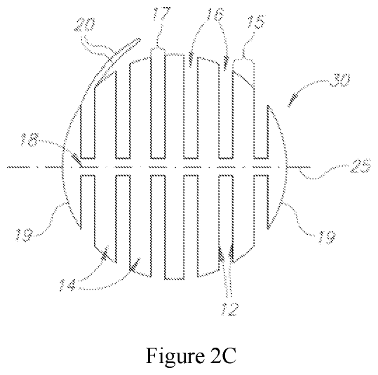

In some embodiments, with reference to FIGS. 2A-B, and as described in WO/2014/037862, published on Mar. 13, 2014, which is incorporated herein by reference in its entirety, grooved carriers 30 are used for proliferation and/or incubation of plant cells. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate. Carriers 30 can include multiple two-dimensional (2D) surfaces 12 extending from an exterior of carrier 30 towards an interior of carrier 30. As shown, the surfaces are formed by a group of ribs 14 that are spaced apart to form openings 16, which may be sized to allow flow of cells and culture medium (not shown) during use. With reference to FIG. 2C, carrier 30 can also include multiple 2D surfaces 12 extending from a central carrier axis 18 of carrier 30 and extending generally perpendicular to ribs 14 that are spaced apart to form openings 16, creating multiple 2D surfaces 12. In some embodiments, carriers 30 are “3D bodies” as described in WO/2014/037862; the contents of which relating to 3D bodies are incorporated herein by reference.

In certain embodiments, the described carriers (e.g. grooved carriers) are used in a bioreactor. In some, the carriers are in a packed conformation.

In still other embodiments, the material forming the multiple 2D surfaces comprises at least one polymer. Suitable coatings may, in some embodiments, be selected to control cell attachment or parameters of cell biology.

Harvesting and/or Lysing of Cannabinoid-Producing Cells

In certain embodiments, the described method further comprises the subsequent step (following the described incubation(s) in one or more media) of harvesting the plant cells by removing them from the 3D matrix. In more particular embodiments, cells may be removed from a 3D matrix while the matrix remains within the bioreactor.

Alternatively, the cells are lysed while attached to the matrix. In certain embodiments, the cells are lysed while attached to a 3D matrix, while within the bioreactor.

In general, embodiments of harvesting the plant cells can be performed in addition to, or in other embodiments without harvesting cannabinoids from the medium in which the cells were incubated.

In certain embodiments, the harvest from the bioreactor is performed when at least about 10%, 12%, 14%, 16%, 18%, 20%, 22%, 24%, 26%, 28%, 30%, 32%, 35%, or in other embodiments at least 40%, of the cells are in the S and G2/M phases (collectively), as can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells.

In still other embodiments, the plant cells are harvested from the bioreactor by a process comprising vibration or agitation, for example as described in PCT International Application Publ. No. WO 2012/140519, which is incorporated herein by reference. In certain embodiments, to effect harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types I, II, III, and IV, with EDTA. Enzymes with similar activity to trypsin are well known in the art; non-limiting examples are TrypLE™, a fungal trypsin-like protease, and Collagenase, Types I, II, III, and IV, which are available commercially from Life Technologies. Enzymes with similar activity to collagenase are well known in the art; non-limiting examples are Dispase I and Dispase II, which are available commercially from Sigma-Aldrich. In still other embodiments, the cells are harvested by a process comprising an optional wash step, followed by incubation with collagenase, followed by incubation with trypsin. In various embodiments, at least one, at least two, or all three of the aforementioned steps comprise agitation. In more specific embodiments, the total duration of agitation during and/or after treatment with protease plus a calcium chelator is between 2-10 minutes, in other embodiments between 3-9 minutes, in other embodiments between 3-8 minutes, and in still other embodiments between 3-7 minutes. In still other embodiments, the cells are subjected to agitation at 0.7-6 Hertz, or in other embodiments 1-3 Hertz, during the wash step before the protease and calcium chelator are added.

In other embodiments, any of the aforementioned vibration or agitation steps are used to lyse the cannabinoid-producing cells within the bioreactor. In certain embodiments, vibration or agitation is performed in the presence of one or more abrasive agents, non-limiting examples of which are beads. In certain embodiments, [Santos A R. et. al]. In certain embodiments, the beads are glass. In other embodiments, the beads comprise a plastic, ceramic, or metal. Non-limiting examples of useful beads are described in US patent appl. pub. no. 2019/0024039 to Phillip Belgrader, which is incorporated by reference herein. Alternatively or in addition, other physical disruption techniques are used, e.g., sonication (Rajagopalan M. et. al).

In still other embodiments, the cells are subject to a detergent treatment (e.g., an ionic detergent) that dissolves lipids on the outer surface of the cell wall, followed by (or, in other embodiments, in conjunction with) enzymatic treatment that digests plant cell walls, a non-limiting example of which is treatment with a muramidase (lysozyme) or achromopeptidase. Strictly for purposes of exemplification, Babu V and Choudhury B used a mixture of sodium cholate and sodium deoxycholate at 0.2% and 0.5%, respectively, for 5 minutes, followed by 2 g/l lysozyme for 1 hour. Non-limiting examples of cell wall lysis methods are described in US patent appl. pub. no. 20130109027 to Gabor Kiss et. al, which is incorporated herein by reference.

Those skilled in the art will appreciate that a variety of isotonic buffers may be used for washing cells and similar uses. Hank's Balanced Salt Solution (HBSS; Life Technologies) is only one of many buffers that may be used.

In other embodiments, the cells are harvested from a 2D matrix.

In yet other embodiments, the cells are harvested from a suspension. In some embodiments, harvest includes vibration or agitation. In other embodiments, harvest includes removing the cell suspension from the growth apparatus or bioreactor and isolating the cells, e.g. by centrifugation.

In some embodiments, the cells are harvested intact, e.g. with at least 60% of the cells intact, or, in other embodiments, at least 65%, 70%, 75%, 80%, 85%, 90%, 95%, or 97% of the cells intact. Following harvesting, the cells are, in some embodiments, lysed by methods known in the art, non-limiting examples of which are those described in Tsugama et al; and CelLytic™ P, available from Sigma-Aldrich at cat. no. C2360; and methods described in US patent appl. publ. no. 2015/0167053, to Thomas R Mertz, JR, which is incorporated herein by reference. Other methods include physical disruption, e.g. mechanical disruption, liquid homogenization, high frequency sound waves, and subjecting the cells to freeze/thaw cycles.

In other embodiments, the cells are lysed while still attached to the described 3D matrix, or, in other embodiments, while attached to the 2D matrix, or, in other embodiments, while in suspension. In more specific embodiments, lysis is performed by methods known in the art, non-limiting examples of which are those described in Tsugama et al; and CelLytic™ P, available from Sigma-Aldrich at cat. no. C2360; and methods described in US patent appl. publ. no. 2015/0167053. Other methods include physical disruption, e.g. mechanical disruption, liquid homogenization, high frequency sound waves, and subjecting the cells to freeze/thaw cycles.

In yet other embodiments, the cells are lysed while still attached to the described 3D matrix, by means of by a process comprising vibration or agitation, for example as described in PCT International Application Publ. No. WO 2012/140519. In certain embodiments, to effect harvesting, the cells are agitated at 3-5 Hertz, or in other embodiments 4-6 Hertz, or in other embodiments 5-7 Hertz, or in other embodiments 6-8 Hertz, or in other embodiments 7-10 Hertz, during, or in other embodiments during and after, treatment with a protease, optionally also comprising a calcium chelator. In other embodiments, a detergent is present during the vibration or agitation. In still other embodiments, beads (e.g. made of glass, steel or ceramic) are present during the vibration or agitation. In more specific embodiments, the beads are 0.25-0.5 mm in diameter.

Following lysis, in some embodiments, cannabinoids are extracted from the cell lysate by methods known in the art.

Relevant Genes and Enzymes

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

In some embodiments, as mentioned, the described plant cells comprise an enzyme or protein involved in the synthesis of one or more cannabinoids (“cannabinoid synthetic enzyme”) or the secretion of one or more cannabinoids (“cannabinoid secretion enzyme”). In certain embodiments, the enzyme is an endogenous enzyme. In other embodiments, the enzyme is an exogenous enzyme. In other embodiments, the cells comprise a gene(s) encoding the described enzyme(s).

In other embodiments, the enzyme is geranyl-pyrophosphate-olivetolic acid geranyltransferase (EC 2.5.1.102), which performs condensation of geranyl pyrophosphate with olivetolic acid to produce cannabigerolic acid (CBGA).

In still other embodiments, the enzyme is THCA synthase (UniprotKM Accession No. A0A0E3XJ68), which performs oxidative cyclization of CBGA to generate tetrahydrocannabinolic acid (THCA). Those skilled in the art will appreciate that, in further embodiments, THCA may be transformed into THC by non-enzymatic decarboxylation.

In other embodiments, the described plant cell expresses (or, in other embodiments, overexpresses) one or more transcription factors that enhance metabolite flux through the cannabinoid biosynthetic pathway, a non-limiting example of which is a Myb protein, non-limiting examples of which are CAN833 and/or CAN738 (Marks M D et al), Myb12, Myb8, AtMyb12, and MYB 112 (Trait).

In some embodiments, the described plant cells comprise cytochrome P450's (CYP) monooxygenases, which are utilized, in more specific embodiments, to transiently modify or functionalize the chemical structure of the cannabinoids to produce water-soluble forms, for example as described in WO2019/014395 to Trait Biosciences, which is incorporated by reference herein.

In still other embodiments, the plant cells express CBDA synthase.

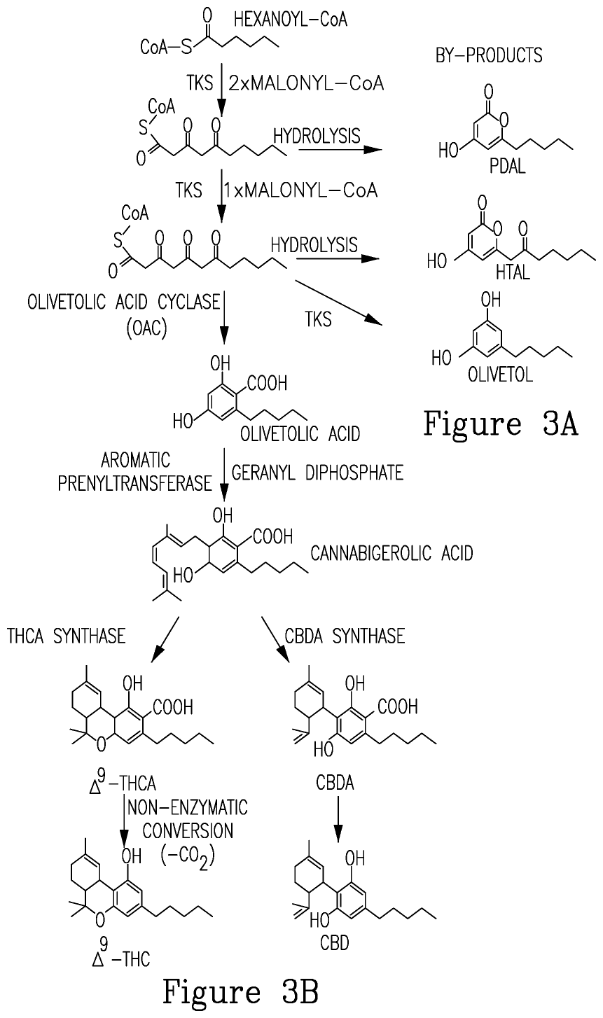

In other embodiments, the plant cells express genes encoding olivetolic acid cyclase, aromatic prenyltransferase, or any of the enzymes in FIG. 3, each of which represents a separate embodiment.

In yet other embodiments, the plant cells express one or more glycosyltransferase enzymes, such as UDP-glycosyltransferase (UGT), to catalyze, in vivo the glucuronosylation or glucuronidation of cannabinoids, such as primary (CBD, CBN) and secondary (THC, JWH-018, JWH-073) cannabinoids. In this embodiment, glucuronidation may consist of the transfer of a glucuronic, for example as described in WO2019/014395 to Trait Biosciences, which is incorporated by reference herein.

In other embodiments, the plant cells express an enzyme for detoxification of hydrogen peroxide, a non-limiting example of which is a Catalase. Those skilled in the art will appreciate, in light of the present disclosure, that the exact Catalase used is not critical. Strictly for exemplification, the sequence of Arabidopsis thaliana catalase is set forth in SEQ ID NO. 13-14 of WO2019/014395 to Trait Biosciences, which is incorporated by reference herein.

In other embodiments, the gene encoding one or more cannabinoid synthases is modified to remove all or part of the N-terminal extracellular targeting sequence. An exemplary trichome targeting sequence for THCA synthase is identified SEQ ID NO. 40, while trichome targeting sequence for CBDA synthase is identified SEQ ID NO. 41. Co-expression with this cytosolic-targeted synthase with a cytosolic-targeted CYP or glycosyltransferase, may allow the localization of cannabinoid synthesis, accumulation and modification to the cytosol. Such cytosolic target enzymes may be co-expressed with catalase, ABC transporter or other genes that may reduce cannabinoid biosynthesis toxicity and or facilitate transport through or out of the cell. Non-limiting examples of these possibilities are described in WO2019/014395 to Trait Biosciences, which is incorporated by reference herein.

Alternatively or in addition, a cannabinoid secretion enzyme is expressed by the plant cell. In some embodiments, the cannabinoid secretion enzyme is a transporter protein, a non-limiting example of which is the ABC transporter, as described in WO2019/014395 to Trait Biosciences, which is incorporated by reference herein.

Each of the described enzymes cannabinoid synthetic enzymes and cannabinoid secretion enzymes may be freely combined with one or more of any of the other described enzymes.

Those skilled in the art will appreciate, in light of the present disclosure, that enhancer elements can be used to confer inducible expression of one or more genes operably linked thereto.

Work-up

In certain embodiments, conditioned media, i.e. post-incubation medium from the described incubation step(s) is subjected to additional steps to isolate and/or modify the desired cannabinoids. As a non-limiting example, THC and CBD may be derived artificially from their acidic precursors tetrahydrocannabinolic acid (THCA) and cannabidiolic acid (CBDA) by non-enzymatic decarboxylation, for example as described in WO2019/014395 to Trait Biosciences, which is incorporated by reference herein. In other embodiments, the work-up comprises the use of glycosidase enzymes, e.g. for removal of one or more sugar moieties from the cannabinoid molecules. Non-limiting examples of such treatment are described in WO2019/014395 to Trait Biosciences, which is incorporated by reference herein.

In other embodiments, the products of the described incubation methods (e.g. harvested cells, or, in other embodiments, cell lysates, or, in other embodiments, fractions of cell lysates) are subject to lyophilization, which may be, in more specific embodiments, freeze drying or spray drying. Method for lyophilizing cells are known to those skilled in the art; non-limiting embodiments of such methods are described in Hussein.

Cannabinoids, CM, Compositions, and Methods of Utilizing Same

In other embodiments, there is provided a population of plant cells treated by the described methods. In other embodiments, there is provided a composition, comprising cannabinoids produced from the cells. In certain embodiments, the composition is a pharmaceutical composition and/or further comprises a pharmacologically acceptable excipient. The cells may be any embodiment of expanded cells mentioned herein, each of which is considered a separate embodiment. In further embodiments, the pharmaceutical composition may be indicated for ameliorating side effects of chemotherapy with cytostatic drugs, alleviation of chronic pain associated with cancer, anti-spastic activity in multiple sclerosis or Tourette's syndrome cases, eating disorders associated with AIDS and anorexia; autism; epilepsy; or inflammatory bowel disease (IBD) (Borrelli et al. 2013; Grotenhermen and Müller-Vahl 2012; Szaflarski and Bebin 2014; Wrobel et al)

In other embodiments, there is provided conditioned medium (CM) derived from the described methods, for example post-incubation medium from the described incubation step(s). In still other embodiments, there is provided CM derived from incubating cells following expansion by the described methods. In yet other embodiments, there is provided a pharmaceutical composition comprising the CM. Those skilled in the art will appreciate that, in certain embodiments, various bioreactors may be used to prepare CM, including but not limited to plug-flow bioreactors, and stationary-bed bioreactors (Kompier R et al. Use of a stationary bed reactor and serum-free medium for the production of recombinant proteins in insect cells. Enzyme Microb Technol. 1991. 13(10):822-7.) For example, CM is produced as a by-product of the described methods for cell expansion and cannabinoid production. The CM in the bioreactor can be removed from the bioreactor or otherwise isolated. In other embodiments, the described expanded cells are removed from the bioreactor and incubated in another apparatus (a non-limiting example of which is a tissue culture apparatus), and CM from the cells is collected.

In still other embodiments, there is provided a culture, comprising the described cells, or in other embodiments a bioreactor, comprising the described culture. Except where indicated otherwise, the term “bioreactor” refers to an apparatus comprising a cell culture chamber and external medium reservoir (a non-limiting example of which is a feed bag) that is operably connected with the cell culture chamber so as to enable medium exchange between the two compartments (perfusion). In some embodiments, the bioreactor further comprises a synthetic material that is a 3D substrate. In other embodiments, the bioreactor further comprises a synthetic material that is a 2D substrate. In still other embodiments, no solid phase growth substrate is present in the bioreactor. The cells may be any embodiment of expanded cells mentioned herein, each of which may be freely combined with the mentioned embodiments of bioreactor conditions.

In still other embodiments, there is provided a suspension comprising any of the described cell populations. In certain embodiments, the suspension comprises a pharmaceutically acceptable excipient. In other embodiments, the suspension is a pharmaceutical composition.

The described cannabinoids can be, in some embodiments, administered as a part of a pharmaceutical composition that further comprises one or more pharmaceutically acceptable carriers. Hereinafter, the term “pharmaceutically acceptable carrier” refers to a carrier or a diluent. In some embodiments, the carrier or diluent does not cause significant irritation to a subject and does not abrogate the biological activity and properties of the administered cells. Examples, without limitations, of carriers are propylene glycol, saline, emulsions and mixtures of organic solvents with water. In some embodiments, the pharmaceutical carrier is an aqueous solution of saline. In other embodiments, the composition further comprises a pharmacologically acceptable excipient. Alternatively or in addition, the composition is frozen.

In other embodiments, compositions are provided herein that comprises cannabinoids in combination with an excipient, e.g., a pharmacologically acceptable excipient. The cannabinoids may be any embodiment of cannabinoids mentioned herein, each of which is considered a separate embodiment.

One may, in various embodiments, administer the pharmaceutical composition in a systemic manner (as detailed hereinabove). In other embodiments, the pharmaceutical composition is formulated for administration by ingestion, for example in a foodstuff. In still other embodiments, the composition is formulated for administration by inhalation, a non-limiting example of which is smoking, e.g. as traditionally done with cannabis products. In yet other embodiments, the composition is formulated for administration with a vaporizer. Alternatively, one may administer the pharmaceutical composition locally, a non-limiting example of which is subcutaneous (SC) administration. In this regard, “subcutaneous” administration refers to administration just below the skin.

In other embodiments, for injection, the described cannabinoids may be formulated in aqueous solutions, e.g. in physiologically compatible buffers such as Hank's solution, Ringer's solution, or physiological salt buffer, optionally in combination with medium containing excipients.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

Depending on the severity and responsiveness of the condition to be treated, dosing can be of a single or, in other embodiments, a plurality of administrations, with a course of treatment lasting from several days to several weeks or, in other embodiments, until alleviation of the disease state is achieved.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

The described cannabinoids are, in some embodiments, suitably formulated as pharmaceutical compositions which can be suitably packaged as an article of manufacture. Such an article of manufacture comprises a packaging material which comprises a label describing a use in treating a symptom, disease, or disorder, for example side effects of chemotherapy, chronic pain associated with cancer, multiple sclerosis, Tourette's syndrome, eating disorders associated with AIDS and anorexia, autism, epilepsy, IBD, or another therapeutic indication mentioned herein. In other embodiments, a pharmaceutical agent is contained within the packaging material, wherein the pharmaceutical agent is effective for the treatment of a hematologic disorder. In some embodiments, the pharmaceutical composition is frozen.

A typical dosage of the described cannabinoids used alone might range, in some embodiments, from about 1-200 mg per day. In other embodiments, the dose is 1-10, 1-20, 1-30, 1-40, 1-50, 1-60, 1-80, 1-100, 1-150, 2-10, 2-20, 2-30, 2-40, 2-50, 2-60, 2-80, 2-100, 2-150, 3-10, 3-20, 3-30, 3-40, 3-50, 3-60, 3-80, 3-100, 3-150, 5-10, 5-20, 5-30, 5-40, 5-50, 5-60, 5-80, 5-100, 5-150, 10-20, 10-30, 10-40, 10-50, 10-60, 10-80, 10-100, 10-150, 10-200, 10-300, 10-400, 10-500, 10-600, 10-800, 10-1000, 10-1500, 15-200, 15-300, 15-400, 15-500, 15-600, 15-800, 15-1000, 15-1500, 20-200, 20-300, 20-400, 20-500, 20-600, 20-800, 20-1000, 20-1500, 30-200, 30-300, 30-400, 30-500, 30-600, 30-800, 30-1000, 30-1500, 50-200, 50-300, 50-400, 50-500, 50-600, 50-800, 50-1000, 50-1500, 70-200, 70-300, 70-400, 70-500, 70-600, 70-800, 70-1000, 70-1500, 100-200, 100-300, 100-400, 100-500, 100-600, 100-800, 100-1000, or 100-1500 mg/day. In still other embodiments, the dose is 0.02-4 mg/kg/day. In other embodiments, the dose is 0.02-3, 0.02-2, 0.02-1.5, 0.02-1, 0.02-0.8, 0.02-0.5, 0.02-0.4, 0.02-0.3, 0.02-0.2, 0.02-0.15, 0.02-1, 0.03-3, 0.03-2, 0.03-1.5, 0.03-1, 0.03-0.8, 0.03-0.5, 0.03-0.4, 0.03-0.3, 0.03-0.2, 0.03-0.15, 0.03-1, 0.05-3, 0.05-2, 0.05-1.5, 0.05-1, 0.05-0.8, 0.05-0.5, 0.05-0.4, 0.05-0.3, 0.05-0.2, 0.05-0.15, 0.05-1, 0.1-3, 0.1-2, 0.1-1.5, 0.1-1, 0.1-0.8, 0.1-0.5, 0.1-0.4, 0.1-0.3, 0.1-0.2, 0.1-0.15, or 0.1-1 mg/kg/day. In certain embodiments, the aforementioned dosages refer to the amount of CBD; in other embodiments, the amount of THC; or in other embodiments, the total amount of cannabinoids.

Subjects and Routes of Administration

In certain embodiments, the subject treated by the described methods and compositions is a human. In other embodiments, the subject may be an animal. In certain embodiments, the subject may be administered with additional therapeutic agents.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including cannabinoids. In another aspect, the kits and articles of manufacture may comprise a label, instructions, and packaging material, for example for treating a symptom, disease, or disorder, e.g. side effects of chemotherapy, chronic pain associated with cancer, multiple sclerosis, Tourette's syndrome, eating disorders associated with AIDS and anorexia, autism, epilepsy, IBD, or for another therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional References are Cited in Text

Aina et al., (2012) “Thidiazuron-Induced Tissue Culture Regeneration from Quartered-Seed Explants of Arachis paraguariensis”. Crop Science. 52 (3): 555.
Aizpurua-Olaizola O et al., (2016) Evolution of the cannabinoid and terpene content during the growth of Cannabis sativa plants from different chemotypes. J Nat Prod 79:324-331
Andre C M, Hausman J-F, Guerriero G (2016) Cannabis sativa: the plant of the thousand and one molecules. Front Plant Sci. 2016 Feb. 4; 7:19.
Babu V and Choudhury B, Methods of cell lysis and effect of detergents for the recovery of nitrile metabolizing enzyme from Amycolatopsis sp. IITR215. J Genet Eng and Biotechnol, 2013, 11(2): 117-122
Borrelli F et al., Beneficial effect of the non-psychotropic plant cannabinoid cannabigerol on experimental inflammatory bowel disease. Biochem Pharmacol. 2013 May 1; 85(9):1306-16.
Braemer R, Paris M (1987) Biotransformation of cannabinoids by a cell suspension culture of Cannabis sativa L. Plant Cell Rep 6 (2):150-152.
Burstein S (2015) Cannabidiol (CBD) and its analogs: a review of their effects on inflammation. Bioorg Med Chem 23:1377-1385.
Citti C, et al., A novel phytocannabinoid isolated from Cannabis sativa L. with an in vivo cannabimimetic activity higher than Δ9-tetrahydrocannabinol: Δ9-Tetrahydrocannabiphorol. Sci Rep 9, 20335 (2019). https://doi.org/10.1038/s41598-019-56785-1
De Petrocellis L, Ligresti A, Moriello A S et al (2011) Effects of cannabinoids and cannabinoid-enriched Cannabis extracts on TRP channels and endocannabinoid metabolic enzymes. Br J Pharmacol 163:1479-1494.
Dominici et al. Minimal criteria for defining multipotent mesenchymal stromal cells. The International Society for Cellular Therapy position statement. Cytotherapy. 2006; 8(4):315-7.
Englund A et al. (2013) Cannabidiol inhibits THC-elicited paranoid symptoms and hippocampal-dependent memory impairment. J Psychopharmacol 27:19-27.
Flores-Sanchez I J et al., (2009). Elicitation studies in cell suspension cultures of Cannabis sativa L. Journal of Biotechnology, 143(2), 157-68.
Furusaki and Takeda. Bioreactors for plant cell culture. Ref Mod Life Sci. 2017;
Gamborg O L et al., (1968). “Nutrient requirements of suspension cultures of soybean root cells”. Experimental Cell Research. 50 (1): 151-158.
Grotenhermen and Müller-Vahl, Dtsch Arztebl Int. 2012 July; 109(29-30):495-501. The therapeutic potential of cannabis and cannabinoids.
Haustein M, Ramer R, Linnebacher M et al (2014) Cannabinoids increase lung cancer cell lysis by lymphokine-activated killer cells via upregulation of ICAM-1. Biochem Pharmacol 92:312-325.
Sayed Hussein Farag Hussein. Cannabinoids production in Cannabis sativa L.: An in vitro approach. Dissertation for Technischen Universitat Dortmund.
Javier Lidoy Logroño, In vitro cell culture of Cannabis sativa for the production of cannabinoids. Bacherlor's thesis for Universitat Autonoma de Barcelona.
Lloyd and McCown (1981). “Commercially-feasible micropropagation of mountain laurel, Kalmia latifolia, by use of shoot-tip culture”. Combined Proceedings, International Plant Propagators' Society. 30: 421-427
Marks M D et al., Identification of candidate genes affecting Δ9-tetrahydrocannabinol biosynthesis in Cannabis sativa. J Exp Bot. 2009 September; 60(13): 3715-3726.
Mustafa et al., (2011). Nature Protocols 6(6): 715-742.
Nagai et al., J Fermentation and Bioengineering. 77(2): 212-214.1994
Pierik R L M (1987) In vitro culture of higher plants. Martinus Nijhoff Dordrecht, the Netherlands.
Pryce G, Riddall D R, Selwood D L et al (2015) Neuroprotection in experimental autoimmune encephalomyelitis and progressive multiple sclerosis by Cannabis-based cannabinoids. J Neuroimmune Pharmacol 10:281-292.
Rajagopalan M. et. al., Lett. Appl. Microbiol., 21 (1995), pp. 14-17.
Rao & Ravishankar (2002). Plant cell cultures: Chemical factories of secondary metabolites. Biotechnology Advances, 20(2), 101-53
Remotti and Loffler (1995). Callus induction and plant regeneration from Gladiolus.Plant Cell Tiss Org 42 (2):171-178.
Ruffoni, B et al., (2010). Plant Cell Cultures: Bioreactors for Industrial Production
Russo E B (2011) Taming THC: potential Cannabis synergy and phytocannabinoid-terpenoid entourage effects. Br J Pharmacol 163:1344-1364.
Santos A R. et. al., J. Med. Microbiol., 39 (1993), pp. 298-304.
Szaflarski and Bebin, Epilepsy Behay. 2014 December; 41:277-82. Cannabis, cannabidiol, and epilepsy—from receptors to clinical response.
Takayama and Akita. Bioengineering aspects of bioreactor application in plant propagation. In: Dutta S, Ibaraki Y, editors. Plan Tissue Culture Engineering. Dordrecht: Springer; 2006, p. 83-100.
Tsugama et al., A rapid chemical method for lysing Arabidopsis cells for protein analysis. Plant Methods. 2011; 7: 22.
Valdiani et al., Bioreactor-based advances in plant tissue and cell culture: challenges and prospects, Critical Reviews in Biotechnology, 39:1, 20-34, 2019.
Vasconsuelo and Boland (2007) Molecular aspects of the early stages of elicitation of secondary metabolites in plants.
Velasco G, Sanchez C, Guzman M (2012) Towards the use of cannabinoids as antitumour agents. Nat Rev Cancer 12:436-444.
White, P R (1939). “Potentially unlimited growth of excised plant callus in an artificial nutrient”. American Journal of Botany. 26 (2): 59-4.
Wrobel T et al., The application of plant in vitro cultures in cannabinoid production. The application of plant in vitro cultures in cannabinoid production
Zhang and Oppenheimer. A simple and efficient method for isolating trichomes for downstream analyses. Plant Cell Physiol. 2004 February; 45(2):221-4.
Zuardi A W, Crippa J A S, Hallak J E C et al (2006) Cannabidiol, a Cannabis sativa constituent, as an antipsychotic drug. Braz J Med Biol Res 39:421-429.

What is claimed is:

1. A method of producing one or more cannabinoid compounds, comprising: incubating plant cells in a production medium, inside a bioreactor, under conditions where said plant cells produce said one or more cannabinoid compounds; thereby producing one or more cannabinoid compounds.

2. The method of claim 1, wherein said production medium comprises an elicitor.

3. The method of claim 1, wherein said plant cells are incubated in said production medium for at least 3 hours.

4. The method of claim 1, preceded by incubating said plant cells in a bioreactor, under conditions fostering expansion of the plant cells.

5. The method of claim 4, wherein said conditions comprise an expansion medium that differs from said production medium.

6. The method of claim 5, wherein said plant cells are incubated in said expansion medium for at least 3 population doublings.

7. The method of claim 1, wherein said plant cells are C. sativa cells.

8. The method of claim 7, wherein said C. sativa cells are trichome cells.

9. The method of claim 8, wherein said trichome cells are in clumps.

10. The method of claim 8, wherein said trichome cells are in a single-cell suspension.

11. The method of claim 1, wherein said plant cells comprise a gene that encodes a cannabinoid synthetic enzyme.

12. The method of claim 11, wherein the gene is operably linked to an inducible promoter.

13. The method of claim 1, wherein said plant cells comprise a gene that encodes a cannabinoid secretion enzyme.

14. The method of claim 13, wherein the gene is operably linked to an inducible promoter.

15. The method of claim 1, wherein said production medium comprises an inducing agent.

16. The method of claim 1, further comprising controlling at least one of pH, temperature, and levels of dissolved oxygen, glucose, lactate, lactate dehydrogenase, NH₃, and glutamate.

17. The method of claim 1, wherein said bioreactor further comprises a synthetic three-dimensional growth substrate.

18. The method of claim 1, further comprising removing said one or more cannabinoid compounds from said production medium on an ongoing basis.

19. A bioreactor, comprising: a plant cell, a production medium, and one or more cannabinoid compounds disposed in said production medium.

20. The bioreactor of claim 19, wherein said production medium comprises an elicitor.

21. The bioreactor of claim 19, wherein said plant cells are C. sativa cells.

22. The bioreactor of claim 21, wherein said C. sativa cells are trichome cells.

23. The bioreactor of claim 22, wherein said trichome cells are in clumps.

24. The bioreactor of claim 22, wherein said trichome cells are in a single-cell suspension.

25. The bioreactor of claim 19, wherein said plant cells comprise a gene that encodes a cannabinoid synthetic enzyme.

26. The bioreactor of claim 19, wherein said plant cells comprise a gene that encodes a cannabinoid secretion enzyme.

27. The bioreactor of claim 19, wherein said production medium comprises an inducing agent.

28. The bioreactor of claim 19, wherein said bioreactor is configured for further comprising controlling at least one of pH, temperature, and levels of dissolved oxygen, glucose, lactate, lactate dehydrogenase, NH₃, and glutamate.

29. The bioreactor of claim 19, wherein said bioreactor further comprises a synthetic three-dimensional growth substrate.

30. The bioreactor of claim 19, wherein said bioreactor is configured for removing said one or more cannabinoid compounds from said production medium on an ongoing basis.
